(19) United States
(12) Patent Application Publication
(10) Pub. No.: US 2010/0280372 A1
(43) Pub. Date: Nov. 4, 2010

(54) Observation device and method

(76) Inventors: Pieter Poolman, Iowa City, IA (US); Gert Johannes Muller, Murphy, TX (US); Blaze Michael Keller, Seaford, NY (US)

Correspondence Address: Pieter Poolman, 43 Lakeview Dr. NE, Iowa City, IA, 52240, US

(21) Appl. No.: 12/772,943

(22) Filed: May 3, 2010

Related U.S. Application Data
(60) Provisional application No. 61/175,020, filed on May 3, 2009.

(51) Int. Cl. A61B 8/00; A61B 5/05

(52) U.S. Cl. 600/437; 600/407

(57) ABSTRACT

An observation device and method for observing a plurality of physiological and/or anatomical parameters is disclosed. The exemplary embodiment of the observation device is portable and handheld so that it may be used in an emergency situation and is field-deployable. The exemplary embodiment includes an image collector for observing, measuring, and/or recording at least one parameter, which parameter may then be used in analysis and diagnosis of the subject's condition or lack thereof.

### CROSS REFERENCE TO RELATED APPLICATIONS

This non-provisional patent application entitled claims priority from provisional U.S. Pat. App. No. 61/175,020 filed on Feb. 3, 2010.

### FIELD OF INVENTION

The subject matter of this application relates to a device and method for observing physiological and/or anatomical parameters in organisms.

### STATEMENT REGARDING FEDERALLY SPONSORED RESEARCH OR DEVELOPMENT

No federal funds were used to develop or create the invention disclosed and described in the patent application.

### REFERENCE TO SEQUENCE LISTING, A TABLE, OR A COMPUTER PROGRAM LISTING COMPACT DISK APPENDIX

Not Applicable

### AUTHORIZATION PURSUANT TO 37 C.F.R. §1.171 (d)

A portion of the disclosure of this patent document contains material that is subject to copyright and trademark protection. The copyright owner has no objection to the facsimile reproduction by anyone of the patent document or the patent disclosure, as it appears in the Patent and Trademark Office patent file or records, but otherwise reserves all copyrights whatsoever.

### BACKGROUND

In the course of diagnosis, recovery, and other settings, it is desirable to detect several different conditions, such as amblyopia, anisocoria, anterior ischemic optic neuropathy, blepharospasm, concussion, drusen, encephalitis, epilepsy, hemifacial spasm, microvascular cranial nerve palsy, migraine, myasthenia gravis, optic neuritis, pituitary tumor, pseudotumor cerebri, seizures, sobriety, stroke, thyroid malfunction, traumatic brain injury, and vision tests, for example, visual acuity, perimetry, and visual useful fields.

Relevant background literature includes:

Chua, K. S.; Ng, Y. S.; Yap, S. G.; Bok, C. W. 2007. A brief review of traumatic brain injury rehabilitation. Annals of the Academy of Medicine, Singapore, 36 (1):31-42.

Ghajar, J.; Ivry, R. B. 2009. The Predictive Brain State: Asynchrony in Disorders of Attention? The Neuroscientist. SAGE Open pre-publication.

Kapoor, N.; Ciuffreda, K. J. 2002. Vision Disturbances Following Traumatic Brain Injury. Current Treatment Options in Neurology, 4 (4):271-280.

Levin, L. A. 2004. Neuro-ophthalmologic diagnosis and therapy of central nervous system trauma. Ophthalmology Clinics of North America, 17 (3):455-64.

Suh, M.; Basu, S.; Kolster, R.; Sarkar, R.; McCandliss, B.; Ghajar, J. 2006. Increased oculomotor deficits during target blanking as an indicator of mild traumatic brain injury. Neuroscience Letters, 410 (3):203-7.

Suh, M.; Kolster, R.; Sarkar, R.; McCandliss, B.; Ghajar, J. 2006. Deficits in predictive smooth pursuit after mild traumatic brain injury. Neuroscience Letters, 401 (1-2):108-13.

Szymanowicz, D. G.; Thiagarajan, P.; Green, W.; Ciuffreda, K. J.; Ludlam, D. 2009. Vergence Dynamics in Traumatic Brain Injury. Proceedings of the ARVO 2009 meeting, Fort Lauderdale.

Zelinsky, D. 2007. Neuro-optometric diagnosis, treatment and rehabilitation following traumatic brain injuries: a brief overview. Physical medicine and rehabilitation clinics of North America, 18 (1):87-107.

Brooks, J.; Fos, L. A., Greve, K. W.; Hammond, J. S. 1999. Assessment of executive function in patients with mild traumatic brain injury. Journal of Trauma, 46:159-163.

Centers for Disease Control and Prevention (CDC), National Center for Injury Prevention and Control. 2003. Report to Congress on mild traumatic brain injury in the United States: steps to prevent a serious public health problem. Centers for Disease Control.

Centers for Disease Control and Prevention (CDC). 1999. Facts about concussion and brain injury, CDC.

Centers for Disease Control and Prevention (CDC). Sports-related recurrent brain injuries—United States. Morbidity and Mortality Weekly Reports, 46: 224-227.

Finkelstein, E.; Corso, P.; Miller, T. 2006. The Incidence and Economic Burden of Injuries in the United States. New York: Oxford University Press.

Fontaine A.; Azouvi, P.; Remy, P.; Bussel, B.; Samson, Y. 1999. Functional anatomy of neuropsychological deficits after severe traumatic brain injury. Neurology, 53:1963-1968.

Goldberg, E.; Bougakov, D. 2005. Neuropsychologic assessment of frontal lobe dysfunction. Psychiatric Clinics of North America, 28:567-580.

Gupta, R. K.; Saksena, S.; Agarwal, A.; Hasan, K. M.; Husain, M.; Gupta, V.; Narayana, P. A. 2005. Diffusion tensor imaging in late posttraumatic epilepsy. Epilepsia, 46:1465-1471.

Hebb, M. O.; McArthur, D. L.; Alger, J.; Etchepare, M.; Glenn, T. C.; Bergsneider, M.; Martin, N.; Vespa, P. M. 2007. Impaired percent alpha variability on continuous electroencephalography is associated with thalamic injury and predicts poor long-term outcome after human traumatic brain injury. Journal of Neurotrauma, 24:579-590.

Inglese, M.; Makani, S.; Johnson, G.; Cohen, B. A.; Silver, J. A.; Gonen, O; Grossman, R. I. 2005. Diffuse axonal injury in mild traumatic brain injury: a diffusion tensor imaging study. Journal of Neurosurgery, 103:298-303.

Ivins, B. J.; Schwab, K.; Warden, D.; Harvey, S.; Hoilien, M.; Powell, J.; Johnson, C. S.; Salazar, A. M. 2003. Traumatic brain injury in U.S. army paratroopers: prevalence and character. Journal of Trauma Injury, Infection and Critical Care, 55:617-621.

Kegler, S.; Coronado, V.; Annest, J.; Thurman, D. 2003. Estimating nonfatal traumatic brain injury hospitalizations using an urban/rural index. Journal of Head Trauma Rehabilitation, 18:469-78.

Langlois, J. A.; Kegler, S. R.; Butler, J. A.; Gotsch, K. E.; Johnson, R. L.; Reichard, A. A.; Webb, K. W.; Coronado, V. G.; Selassie, A. W.; Thurman, D. J. 1997. Traumatic brain injury-related hospital discharges: results from a fourteen state surveillance system. Morbidity and Mortality Weekly Reports, 52:1-20.

Langlois, J.; Marr, A.; Mitchko, J.; Johnson, R. 2005. Tracking the silent epidemic and educating the public: CDC's traumatic brain injury-associated activities under the TBI Act of 1996 and the Children's Health Act of 2000. Journal of Head Trauma Rehabilitation, 20:196-204.

Langlois, J. A.; Rutland-Brown, W.; Thomas, K. E. 2004. Traumatic brain injury in the United States: emergency department visits, hospitalizations, and deaths. Centers for Disease Control.

Lew, H. L.; Poole, J. H.; Castaneda, A.; Salerno, R. M.; Gray, M. 2006. Prognostic value of evoked and event-related potentials in moderate to severe brain injury. Journal of Head Trauma Rehabilitation, 21:350-360.

McDonald, B. C.; Flashman, L. A.; Saykin, A. J. 2002. Executive dysfunction following traumatic brain injury: neural substrates and treatment strategies. NeuroRehabilitation, 17:333-344.

National Institute of Neurological Disorders and Stroke. 2002. Traumatic brain injury: hope through research. National Institutes of Health; NIH Publication No.: 02-158.

Parrish, J.; Geary, E.; Jones, J.; Seth, R.; Hermann, B.; Seidenberg, M. 2007. Executive functioning in childhood epilepsy: parent-report and cognitive assessment. Developmental Medicine & Child Neurology, 49:412-426.

Plata, C. M.; Ardelean, A.; Koovakkattu, D.; Srinivasan, P.; Miller, A.; Phuong, V.; Harper, C.; Moore, C.; Whittemore, A.; Madden, C.; Diaz-Arrastia, R.; Devous, M. 2007. Magnetic resonance imaging of diffuse axonal injury: quantitative assessment of white matter lesion volume. Journal of Neurotrauma, 24:591-598.

Rapoport, M. J.; McCullagh, S.; Shammi, P.; Feinstein, A. 2005. Cognitive impairment associated with major depression following mild and moderate traumatic brain injury. Journal of Neuorpsyhiatry & Clinical Neuroscience, 17, 61-65.

Seal, K. H.; Bertenthal, D.; Miner, C. R.; Sen, S.; Marmar, C. 2007. Bringing the war back home: mental health disorders among 103,788 US veterans returning from Iraq and Afghanistan seen at Department of Veterans Affairs facilities. Archives of Internal Medicine, 167:467-482.

Shiga, T.; Ikoma, K.; Katoh, C.; Isoyama, H.; Matsuyama, T.; Kuge, Y.; Kageyama, H.; Kohno, T.; Tarae, S.; Tamaki, N. 2006. Loss of neuronal integrity: a cause of hypometabolism in patients with traumatic brain injury without MRI abnormality in the chronic stage. European Journal of Nuclear Medicine and Molecular Imaging, 33:817-822.

Thurman, D.; Alverson, C.; Dunn, K.; Guerrero, J.; Sniezek, J. 1999. Traumatic brain injury in the United States: a public health perspective. Journal of Head Trauma Rehabilitation, 14:602-615.

Warden, D. 2006. Military TBI during the Iraq and Afghanistan wars. Journal of Head Trauma Rehabilitation, 21:398-402.

U.S. Pat. Nos. 5,943,954; 5,956,125; 6,659,611; 7,261690; 7,319,780; and 7,488,294.

U.S. Pat. Pub. No. 2009/0018407.

All the above-referenced prior art publications are incorporated herein by reference in their entirety.

Traumatic brain injury (TBI) is an especially salient condition that requires proper diagnosis and treatment immediately after the triggering event. TBI results from a significant blow or jolt to the head or a penetrating injury that disrupts the functioning of the brain. Severity of damage to the brain ranges from “mild” symptoms such as a brief change in mental status or consciousness to a “severe” injury, including an extended period of unconsciousness or amnesia (CDC, 1999).

TBI has been implicated by the World Health Organization to be a 21st century epidemic similar to malaria and HIV/AIDS, not restricted to the developed world. One third of patients may suffer severe TBI with long-term cognitive and behavioral disabilities. Injuries to the brain do not only damage the cerebrum but may give rise to a multisystem disorder due to associated injuries in 20% of cases, which can include complex neurological impairments, neuroendocrine and neuromedical complications. There is promising evidence of improved outcome and functional benefits with early induction into a trans-disciplinary brain injury rehabilitation program. However, TBI research is fraught with difficulties because of an intrinsically heterogeneous population due to age, injury severity and type, functional outcome measures and small samples (Chua et al., 2007).

Generally, three major modalities are available to assist in the diagnosis, etc. of many nervous system disorders and conditions (e.g., brain injury, stroke, etc.), which include: (1) ultrasonography (acoustic), which provides mostly anatomical measurements; (2) video-based (electromagnetic spectrum), which provides mostly physiological measurements; and, (3) electrophysiology, which provides mostly physiological measurements.

### Scope

As the recent focus and subject of a position statement by the American Academy of Neurology suggests, Traumatic Brain Injury is on the rise in the United States (Warden, 2006). With increased automobile usage and accidents, and with increased deployments of soldiers to hostile environments, many more cases of TBI are being reported than in any previous period. With the increase of emergency medical personnel to triage wounded patients, and with preventative safety measures including airbags in automobiles and body armor for soldiers, many trauma patients often survive wounds that would have previously been fatal. Unfortunately, many patients who survive their traumas do not return to their previous quality of life because they have additionally suffered brain injuries, with deficits ranging from mild sensory loss, to severe cases including symptoms such as paralysis and amnesia.

Falls account for the largest percentage, 28%, of all Traumatic Brain Injuries (Langlois, Rutland-Brown, & Thomas, 2004). Children from birth to 4 years old and adults over the age of 75 are at greatest risk of TBI. Injuries due to motor vehicle accidents (MVA) account for 20% of TBI. Third place is due to injuries from some type of striking event, at 19%. Some 11% of TBI are due to assaults. The rest of the incidents are accounted for by pedal cycle (3%), other transportation events (2%), and suicide attempts (1%) (Langlois, Rutland-Brown, & Thomas, 2004).

Overall, gunshot wounds (GSW) are the leading cause of death related to TBI (CDC, 1999). However, blasts are the leading cause of TBI among active duty military personnel in war zones (CDC, 1999). Certain military duties are more or less related to death rate from TBI, with paratroopers ranking the highest (Ivins et al., 2003). With regard to the recent military activity, in July 2004 a San Francisco Chronicle survey estimated that as many as ⅔ of all OIF/OEF wounded troops processed through Walter Reed suffered some degree to brain injury (APA Monitor, 2005).

### Prevalence

While it is difficult to estimate the prevalence of TBI, the estimates, even the conservative ones, indicate that the prevalence of TBI in the United States is quite high (CDC, 2003; Langlois, Rutland-Brown, & Thomas, 2004). Roughly 1.4 million individuals sustain a brain injury each year. Approximately 50,000 of the cases are fatal, 235,000 patients are hospitalized, and the overwhelming majority of patients are treated and released after a short time in the emergency department (CDC, 2003; Langlois, Rutland-Brown, & Thomas, 2004).

Among children between the ages of 0-14 years, TBI is the cause of approximately 2685 fatalities, 37,000 hospitalizations, and 435,000 emergency department visits each year (CDC, 2003; Langlois, Rutland-Brown, & Thomas, 2004). Again, these estimates are difficult to gauge because many patients are never seen in an emergency department or may not receive any care. In the fatal cases of TBI, the cause of death may often be misattributed to other life-threatening injuries.

When considering the prevalence of TBI, males are almost twice as likely as females to suffer an injury (Langlois, Rutland-Brown, & Thomas, 2004). The two groups at highest risk for TBI are those ages 0 to 4 and also 15 to 19 years of age. However, it is adults 75 years of age or older that are at highest risk of TBI hospitalization and death. Ethnically, African Americans have the highest rate of death from TBI and hospitalization rates are highest among African Americans and American Indians/Alaskan Natives (Langlois et al., 1997).

### Diagnosis & Assessment

Traumatic Brain Injury is often poorly diagnosed by first responders, and it is also considered secondary to the loss of other life sustaining organs, so it is often overlooked as a diagnosis until the patient has been stabilized, medically. The diagnosis, especially of more mild forms of TBI, is often made during a patient's recovery from the acute stages of trauma, when he or she may have regained consciousness and family or care providers recognize that there have been cognitive or other deficits that are the result of the trauma. However, for many patients with more mild forms of TBI, they may even recover and return to their jobs before they start recognizing symptoms of TBI (Inglese et al., 2005; Plata et al., 2007).

If the patient is unconscious or only partially conscious, the Glasgow Coma Scale and Glasgow Outcome Scale are the most common tests used for initial assessment in these acute cases. However, patients with more mild, subtle forms of TBI may go for weeks or months undiagnosed, including symptoms of headaches or neck pain, difficulty remembering or concentrating, slowness in thinking or reading, getting disoriented or being easily confused, anergia, somnolence, sleep deficits, mood changes, dizziness or light-headedness, nausea, sensitivity to lights, ringing in the ears, changes in performance at work or school (CDC, 1999).

### Clinical Outcomes

Approximately 5.3 million Americans have long-term or lifelong deficits in their activities of daily living (ADL) due to Traumatic Brain Injuries (Thurman et al., 1999). Deficits range from mildly annoying symptoms that persist, such as headaches, to catastrophic losses that render the patient unable to manage continence and other loss of physical coordination. Additionally, many patients experience emotional difficulties, including aggression, outbursts, depression, anxiety, PTSD, and other personality changes that cause the person to behave differently in social situations (NINDS, 2002).

Unfortunately, patients who have sustained a TBI are at greater risk for subsequent injuries, including TBI. This risk is elevated for patients with sports injuries, including soccer, boxing, and other head contact oriented activities (CDC, 1997).

Some tests for diagnosing the various conditions listed above have been developed in the prior art, a sample of which are listed herein. Most often, these tests measure and/or observe head, eye, facial, pupil, and/or torso position as well as changes and/or rate of change of these parameters.

TBI commonly impacts on the connections and interactions between signals from sensory, cognitive, motor and emotional systems and signals transmitted via both visual and non-visual retinal fiber pathways (Zelinsky, 2007). The non-visual retinal pathways are actively involved in aspects of living, such as spatial orientation, auditory localization, circadian rhythm and motor function. Non-visual retinal signal processing and linkage dysfunctions require more than specialized neuro-ophthalmologic or traditional eye care evaluation. Neuro-optometric techniques are necessary to test the complex, often overlooked interrelationships among these systems. As part of a multi-disciplinary approach, neuro-optometric intervention is an essential consideration for the optimal diagnosis, treatment and rehabilitation following a TBI.

There is a natural tendency for the physician treating a patient who has sustained trauma to focus on those aspects of the injury that are most visible or apparent (Levin, 2004). Nevertheless, injury to the afferent and efferent visual systems is often concealed, and only a combination of careful examination and judicious use of neuroimaging will lead to the initiation of appropriate management.

Vision disturbances following TBI are common, seen in 30% to 85% of cases. These symptoms are often one of the last to be treated and often escapes detection. Vision disturbances include anomalies of (Kapoor and Ciuffreda, 2002):
Accommodation
Version
Vergence (nonstrabismic, as well as strabismic)
Photosensitivity
Visual field integrity
Ocular health, etc.

On a higher cognitive level, TBI may also lead to attention disorders (Ghajar and Ivry, 2009). Numerous research groups are developing tests and tasks to evaluate vision disturbances as well as deficiencies in attention. Each testing paradigm focuses on a particular visual system feature that might be impacted by TBI. These diagnostic measures are evaluated for sensitivity and specificity during clinical trials.

A wide variety of symptoms can occur after TBI. The nature of the symptoms depends, in large part, on where the brain has been injured. Below is a list of possible physical and cognitive symptoms which can arise from damage to specific areas of the brain (http://www.braininjury.com/symptoms.html), with possible symptoms observable from head, facial, eye, and pupil features highlighted as bold:
Frontal Lobe: Forehead
   Loss of simple movement of various body parts (Paralysis).
   Inability to plan a sequence of complex movements needed to complete multi-stepped tasks, such as making coffee (Sequencing).
   Loss of spontaneity in interacting with others.
   Loss of flexibility in thinking.
   Persistence of a single thought (Perseveration).
   Inability to focus on task (Attending).
   Mood changes (Emotionally Labile).
   Changes in social behavior.
   Changes in personality.
   Difficulty with problem solving.
   Inability to express language (Broca's Aphasia).
Parietal Lobe: near the back and top of the head
   Inability to attend to more than one object at a time.
   Inability to name an object (Anomia).
   Inability to locate the words for writing (Agraphia).
   Problems with reading (Alexia).
   Difficulty with drawing objects.
   Difficulty in distinguishing left from right.
   Difficulty with doing mathematics (Dyscalculia).
   Lack of awareness of certain body parts and/or surrounding space (Apraxia) that leads to difficulties in self-care.
   Inability to focus visual attention.
   Difficulties with eye and hand coordination.
Occipital Lobes: most posterior, at the back of the head
   Defects in vision (Visual Field Cuts).
   Difficulty with locating objects in environment.
   Difficulty with identifying colors (Color Agnosia).
   Production of hallucinations.
   Visual illusions—inaccurately seeing objects.
   Word blindness—inability to recognize words.
   Difficulty in recognizing drawn objects.
   Inability to recognize the movement of object (Movement Agnosia).
   Difficulties with reading and writing.
Temporal Lobes: side of head above ears
   Difficulty in recognizing faces (Prosopagnosia).
   Difficulty in understanding spoken words (Wernicke's Aphasia).
   Disturbance with selective attention to what we see and hear.
   Difficulty with identification of, and verbalization about objects.
   Short term memory loss.
   Interference with long term memory.
   Increased and decreased interest in sexual behavior.
   Inability to categorize objects (Categorization).
   Right lobe damage can cause persistent talking.
   Increased aggressive behavior.
Brain Stem: deep within the brain
   Decreased vital capacity in breathing, important for speech.
   Swallowing food and water (Dysphagia).
   Difficulty with organization/perception of the environment.
   Problems with balance and movement.
   Dizziness and nausea (Vertigo).
   Sleeping difficulties (Insomnia, sleep apnea).
Cerebellum: base of the skull
   Loss of ability to coordinate fine movements.
   Loss of ability to walk.
   Inability to reach out and grab objects.
   Tremors.
   Dizziness (Vertigo).
   Slurred Speech (Scanning Speech).
   Inability to make rapid movements.

Pupillometry provides useful information about the correlation between light levels and the pupil light reflex to evaluate photosensitivity. According to Levin (2004), examination of the pupils should be conceptualized as being divided into two parts—one looking for afferent pupillary defects and the other for efferent pupillary defects. Afferent pupillary defects result from problems with the transmission of light impulses from the retina along the optic nerve to the pretectal nuclei within the midbrain. Such defects usually result from optic nerve problems but can also be seen with large retinal lesions or (rarely) contralateral optic tract lesions. Efferent pupillary defects result from abnormalities of transmission after the pretectal nuclei of the midbrain send crossed and uncrossed fibers to synapse bilaterally on the Edinger-Westphal nuclei of the third nerve. An ipsilateral efferent pupillary defect can result from disruption of the input to one of the Edinger-Westphal nuclei, the output fibers along the course of the third nerve, the synapse within the intraorbital ciliary ganglion, or the fibers supplying the pupilloconstrictor fibers within the iris. As a rule of thumb, the presence of unequal size pupils (anisocoria) suggests an efferent pupillary defect, whereas the finding of equal pupils that change size as the eyes are alternately illuminated indicates an afferent pupillary defect. The most commonly missed disorder of the afferent visual system, namely, traumatic optic neuropathy, will be missed with inspection of pupil size alone, because the pupils will be equal in size. In addition, unless there is complete loss of transmission along one or both optic nerves, the pupils will typically react to light. It is the asymmetric reaction to light that is important.

The most efficient way to detect neuro-ophthalmologic disease in the setting of central nervous system trauma is to first examine the pupils in dim light and bright light, looking for anisocoria. If anisocoria is noted, the reactivity of each pupil should be noted individually, as well as disparity of the pupils in the dark versus the light. Following this examination, the swinging flashlight test should be performed, looking for an afferent pupillary defect. This simple three-step test usually detects the most important causes of pupillary abnormalities.

Contusion, shear, or transection damage to the third, fourth, and sixth cranial nerves is often seen after head trauma. A motility examination can be performed rapidly by observing versions (the movement of both eyes in the various positions of gaze) and determining the presence of a tropia (a latent deviation between the position of the two eyes). The most common extraocular motility abnormalities associated with specific cranial nerve palsies should be familiar and include decreased abduction in a sixth nerve palsy, decreased adduction, elevation, and depression with a third nerve palsy, and decreased depression in an adducted position with a fourth nerve palsy. Sometimes the eyes appear to move normally, and it is only the patient's complaints of diplopia that raise the question of abnormal motility. The presence of an exotropia (eyes pointing outward) or esotropia (eyes pointing inward) is often indicative of a problem with adduction or abduction, respectively.

Based on simultaneous independent eye movement tracking, an objective measure of vergence can be obtained. Recent retrospective studies have found significantly slowed dynamic vergence responses in TBI patients. Szymanowicz et al. (2009) have tested non-strabismic TBI patients with near vision symptoms, and asymptomatic visually-normal (non-TBI) subjects. Subjects altered bifixation between two targets every 3-4 seconds upon command, while binocular horizontal position of the eyes was recorded. Targets comprised of a red fixation LED located at 1 m and a white LED placed at 0.3 m in front of the subject, both aligned along the midline. Mean amplitude, time constant, and peak velocity of both convergence and divergence responses of the TBI and normal groups were compared. Although differences in mean convergence and divergence response amplitudes were not significantly different, there were significantly reduced (p<0.01) mean peak velocities and increased time constants for both convergence (15.5±0.94°/sec; 432.4±31 ms) and divergence (15.6±0.96°/sec; 448.8±39.5 ms) in the TBI group when compared to convergence (28.7±1.2°/sec; 221±9.6 ms) and divergence (24.8±1.2°/sec; 273.4±19.1 ms) in the visually-normal group, respectively. Since neural control for vergence involves numerous motor and premotor areas, it appears that multiple axonal pathways are susceptible to diffuse axonal injury from the TBI, resulting in slowed vergence responsivity. However, the similar response amplitudes in both groups suggest unaffected activity of vergence tonic cells related to vergence angle.

In order to evaluate normal levels of attention, Ghajar and Ivry (2009) have used circular smooth pursuit eye movements to measure target-eye position variability. In this test, subjects view a red dot on a computer screen moving in a circle at 0.4 Hz (completing a full circle in 2.5 seconds), while monitored with a 500 Hz infrared camera eye-tracking system. Using this technique, researchers have correlated measures of variability related to the difference in eye-target position and velocity with performance on a working memory task in normal and TBI subjects (Suh, Basu, et al., 2006; Suh, Kolster, et al., 2006). Ghajar and Ivry proposed that this increased variability can originate from damage to the prefrontal cortex, parietal cortex, the cerebellum, or to the fiber tracts that link these regions. The inability to efficiently and consistently use predictive mechanisms is hypothesized to be a major cause of disability in attention disorders. High variability leads to distractibility (and, as a consequence, decreased awareness) and reduced cognitive performance.

### Ultrasonography

Compact, portable and easy to use tools for rapidly diagnosing TBI, as well as other disorders and conditions, are in great demand. As with many other disorders and conditions that are ‘closed brain’ injuries, a significant portion of combat-related TBI results from exposure to blast-related concussive forces that leave little or no external markers of the internal brain injuries. Consequently, these injuries often go undiagnosed until long after the traumatic event, with debilitating and life-threatening symptoms occurring days, weeks or even years following the primary brain injury (Zoroya, 2008). This ‘secondary damage’ results from biochemical processes that activate within minutes or hours after the primary injury (Park et al., 2008; Xiong et al., 2001), underscoring the importance of detecting TBI as soon after primary injury as possible in order to prevent long term damage or even death. While computed axial tomography (CT) scans are considered the ‘gold standard’ for detecting head related injuries, CT scanning technology is difficult to use in the combat environment, given the large infrastructure requirements for operation (power, technicians, physical footprint).

An alternative diagnostic method exploits the changes in intracranial dynamics that result from the concussive forces that lead to TBI. Specifically, elevated intracranial pressure (ICP) is often the most direct, initial result of concussive force related head injuries (Eisenberg et al., 1990). Because of the unique neuro-anatomy of the eye, elevated ICP results in dilation of the optic nerve sheath, increasing its diameter (Gass et al., 1996; Blaivas et al., 2003). Ultrasonographic methods, applied directly—and externally—over either or both eyes, reliably detect these changes (Hansen & Hemke, 1997; Goel et al., 2008; Tayal et al., 2007) and provide a novel and more efficient approach to rapidly identifying deep or bilateral traumatic brain injuries compared to other currently available technologies.

Mixed-technology solutions that combine additional detection capabilities with the ultrasound-based approach could improve either diagnostic sensitivity, specificity, or both. As a result, as embodiment includes an ultrasound-based sensor for detecting elevated ICP resulting from TBI or other conditions, and has a form factor similar to a standard hand-held personal data assistant. The device should function across the wide range of harsh operating environments typical of combat, and should be intuitive and easy to use by medical technicians working under conditions of high stress typical of battlefield environments.

Relevant publications related to ultrasonography, all of which are incorporated by reference herein in their entirety include:

Zoroya, G. 2008. “Delayed TBI diagnoses inspire a mission”. Army Times.

http://www.armytimes.com/news/2008/07/gns_tbi_{—}072408/, 16 Oct. 2008.

Park, E.; Bell, J. D.; Baker, A. J. 2008. Traumatic brain injury: Can the consequences be stopped? Canadian Medical Association Journal, 178 (9):1163-70.

Xiong, Y.; Lee, C. P.; Peterson, P. L. 2001. Mitochondrial dysfunction following traumatic brain injury. In Head Trauma: Basic, Preclinical, and Clinical Directions. Miller L P and Hayes R L, (Eds). John Wiley and Sons, Inc. New York. Pages 257-280.

Eisenberg, H. M.; Gary, H. E. Jr; Aldrich, E. F.; et al. 1990. Initial CT findings in 753 patients with severe head injury. A report from the NIH Traumatic Coma Data Bank. Journal of Neurosurgery, 73:688-98.

Gass, A.; Barker, G. J.; Riordan-Eva, P.; et al. 1996. MRI of the optic nerve in benign intracranial hypertension. Neuroradiology, 38 (8):769-773.

Blaivas, M.; Theodoro, D. L.; Sierzenski, P. 2003. Elevated intracranial pressure detected by bedside emergency ultrasonography of the optic nerve sheath. Academic Emergency Medicine, 10:376-81.

Hansen, H. C.; Helmke, K. 1997. Validation of the optic nerve sheath response to changing cerebrospinal fluid pressure: ultrasound findings during intrathecal infusion tests. Journal of Neurosurgery, 87:34-40.

Goel, R. S.; Goyal, N. K.; Dharap, S. B.; Kumar, M.; Gore, M. A. 2008. Utility of optic nerve ultrasonography in head injury. International Journal of the Care of the Injured, 39:519-524.

Tayal, V. S.; Neulander, M.; Norton, H. J.; Foster, T.; Saunders, T.; Blaivas, M. 2007. Emergency Department Sonographic Measurement of Optic Nerve Sheath Diameter to Detect Findings of Increased Intracranial Pressure in Adult Head Injury Patients. Annals of Emergency Medicine, 49 (4):508-514

### Diagnostics Based On Electrical Brain Activity

Electrophysiology may be used to measure brain activity in various ways. Seizures may develop immediately after an injury to the brain or may in delayed fashion, showing up months or years after the initial trauma. Generally speaking, the risk of post traumatic seizures is related to the severity of the injury (http://www.braininjury.com/seizuresandheadinjury.html). Even mild to moderate injuries can result in seizures. It is thought that a head injury disrupts the pathways of the brain and that an epileptic seizure can be viewed as a sort of short circuit of the brain's electrical functioning. Persons who have had head trauma are twelve times as likely as the general population to suffer seizures (Willmore, 1992). Patients with acute intra cranial hematomas also have a high rate of epilepsy. While there are contradictory studies, the more recent study (Lee, 1992) showed that of 4,232 persons suffering mild closed head injury, 53% had early post-traumatic epilepsy. Approximately 57% of head injured individuals developed epilepsy within one-year of injury. Longer onset epilepsy beginning more than four years after the trauma occurs in 20% of patients who developed epilepsy. It is estimated that 30% of all individuals suffering head trauma developed post-traumatic seizures and 80% of the time they occur within the first 24-months (Bakay, 1980).

The primary tool used to diagnose epilepsy is the Electroencephalograph (EEG), which is a device that captures and plots the electrical activity of the brain. Normally, the resting brain emits signals at a frequency of 8 to 13 Hz, called alpha activity, which is best seen in the occipital regions. Theta and delta activity occur in the normal brain as the patient descends into sleep. If the patient is awake, any slowing of electrical activity in a focal area of the brain may indicate a lesion there. Similarly, widespread slowing indicates a widespread disturbance of brain function, often due to a bloodborne insult like low blood sugar, drug intoxication, liver failure, etc. Brain wave activity varies throughout the day depending on the state of alertness. Each area of the brain normally spends a characteristic amount of time in alpha, beta, theta, and delta activity. By comparing the patient's map with that of a control population, it is possible to localize areas of focal slowing of electrical activity. Alone, this type of EEG analysis is insufficient to diagnose brain damage but in conjunction with other neurologic tests, it can be confirmatory.

Evoked studies take advantage of the fact that each time a sensory system of the body—vision, hearing, touch—is stimulated, an electrical signal is generated in the brain. These electrical signals can be detected with electrical wires on the scalp. Thus, visual evoked recordings (VER) are recorded over the occipital lobes; brainstem auditory evoked recordings (BAER) over the temporal lobes; and somatosensory potentials (SSEP) over the parietal lobes.

In terms of EEG monitoring, recently developed sensor technologies aim at improving the ease of practical use of these monitoring systems. Currently, clinical applications of EEG sensors still rely on some form of skin preparation to ensure adequate signal quality, although improper preparation can cause skin irritation, pain or even infection. Using electrolytic gel on the skin tissue is not only uncomfortable and inconvenient, but also causes irritation and swelling during long-term measurement. Furthermore, the conductivity of electrolytic gel decreases gradually due to drying. The decreasing conductance can in turn degrade signal quality. Sigmed has recently introduced the use of MEMS technology based on a silicon-based spiked electrode array or so-called dry electrode array, to enable EEG, EOG, and ECG monitoring without conductive paste or scalp preparation. The dry MEMS sensors feature a microprobe array structure and self-stabilization capability. Microprobe arrays placed on the skin provide signal transmission performance superior to that of standard wet electrodes. Transmission is improved because the electrically conductive probe can penetrate the high electrical resistance of the outer stratum corneum (SC) skin layer and into the deeper stratum germinativum (SG) skin layer, which has better electrical conductivity. The electrically conductive probes are designed to avoid the underlying dermal layer so as to avoid any pain and bleeding.

### Diagnostics Based On Integrated Measures

In order to significantly improve successful diagnosis of TBI, we propose to combine and integrate numerous physiological and neural sensors into a modular system, and to fuse the collected data with other types of clinical diagnoses, apply sophisticated signal processing and classification techniques to gain a fuller picture of patient state. Sensors of interest include EEG, ECG, oximetry, plethysmography, respiration, gas analysis, and head and eye tracking.

We also propose to incorporate many existing and tested algorithms into our framework to process psychophysiological signals. A prime example is EEGLAB. It is an interactive open source Matlab toolbox for processing continuous and event-related EEG, MEG and other electrophysiological data using data averaging, independent component analysis (ICA), time/frequency analysis, artifact rejection, inversion solution, dipole modeling and several modes of data visualization. EEGLAB has been well received and appreciated by the communities of cognitive and clinical neuroscience.

Researchers are constantly developing and patenting new approaches to measure cognitive effort. An example is the Index of Cognitive Activity (ICA) metric, developed by Dr. Sandra Marshall (see www.eyetracking.com). It is well known that effortful cognitive processing is accompanied by increases in pupil dilation, but measurement techniques were not previously available that could supply results in real-time or deal with data collected in long-lasting interactions. The ICA metric, computed in real-time as the operator interacts with the interface, is now available to examine extended periods of usage or to assess critical events on an individual-by-individual basis.

An example of a complete signal analytic procedure for real-time classification is discussed by Poolman et al. (2008). The method utilizes a framework consisting of multiple processing modules that can be applied in whole or in part, including noise mitigation, source-space transformation, discriminant analysis, and performance evaluation. The framework introduces an enhanced noise mitigation technology based on Directed Components Analysis (DCA) that improves upon existing spatial filtering techniques. Source-space transformation, utilizing a finite difference model (FDM) of the human head, estimates activity measures of the cortical sources involved in task performance. Such a source-space discrimination provides measurement invariance between training and testing sessions and holds the promise of providing a degree of classification not possible with scalp-recorded EEG. The framework's discrimination modules interface with performance evaluation modules to generate classification performance statistics.

### Sobriety Testing And Law Enforcement (Adapted From http://www.nhtsa.dot.gov/people/injury/enforce/nystagmus/hgntxt.html#one)

Nystagmus is an involuntary jerking or bouncing of the eyeball that occurs when there is a disturbance of the vestibular (inner ear) system or the oculomotor control of the eye. Horizontal gaze nystagmus (HGN) refers to a lateral or horizontal jerking when the eye gazes to the side. In the impaired driving context, alcohol consumption or consumption of certain other central nervous system depressants, inhalants or phencyclidine, hinders the ability of the brain to correctly control eye muscles, therefore causing the jerk or bounce associated with HGN. As the degree of impairment becomes greater, the jerking or bouncing, i.e. the nystagmus, becomes more pronounced. This is assessed in the horizontal gaze nystagmus test.

Most types of nystagmus, including HGN, are involuntary motions, meaning the person exhibiting the nystagmus cannot control it. In fact, the subject exhibiting the nystagmus is unaware that it is happening because the bouncing of the eye does not affect the subject's vision.

The horizontal gaze nystagmus test is one of three field sobriety tests that comprise the standardized field sobriety test (SFST) battery (the other two tests are the walk-and-turn (WAT) test and the one-leg-stand (OLS) test). Scientific evidence establishes that the horizontal gaze nystagmus test is a reliable roadside measure of a person's impairment due to alcohol or certain other drugs.

Despite the strong correlation between alcohol consumption and HGN, some trial courts across the country still do not admit the results of the HGN test into evidence. Although the scientific evidence to prove this correlation exists, due to lack of knowledge, inadequate preparation, or limited proffers, the evidence prosecutors have presented to courts has at times been insufficient to satisfy the courts' evidentiary standards for admitting scientific or technical evidence. As a result, law enforcement officers in a number of jurisdictions use the HGN test only for purposes of establishing probable cause if at all, without securing admission of the test results into evidence at trial. Ultimately, the fact finder never hears the results of the most reliable field sobriety test.

Legal and law enforcement communities need to better understand that HGN is the most reliable and effective indicator of alcohol impairment and that ample evidence is available to prove that reliability. The challenge is in conveying the strong correlation between the HGN test and impairment to the fact finder and showing how to effectively use the available evidence to prove the HGN test's validity and reliability in court.

Gaze nystagmus is a type of jerk nystagmus where the eye gazing upon or following an object begins to lag and has to correct itself with a saccadic movement toward the direction in which the eye is moving or gazing. Gaze nystagmus is due to disruptions within the nervous system. Alcohol gaze nystagmus (AGN) is gaze nystagmus caused by alcohol. AGN occurs as the eye moves from looking straight ahead (called resting nystagmus), to the side (called HGN), or up (called vertical nystagmus or VGN). The effect of alcohol on eye movement has been described as follows: Alcohol is a central nervous system depressant affecting many of the higher as well as lower motor control systems of the body. This results in poor motor coordination, sluggish reflexes, and emotional instability. The part of the nervous system that fine-tunes and controls hand movements and body posture also controls eye movements. When intoxicated, a person's nervous system will display a breakdown in the smooth and accurate control of eye movements.

This breakdown in the smooth control of eye movement may result in the inability to hold the eyes steady, resulting in a number of observable changes of impaired oculomotor functioning.

### Development of the Standardized Field Sobriety Test Battery

Law enforcement officers have used field sobriety tests (FST) to detect impairment and to develop probable cause to arrest. Most FSTs test coordination, balance and dexterity, all of which diminish as a person reaches higher and higher Blood alcohol contents (BACs). Many FSTs also test a person's ability to perform simple tasks simultaneously because impairment limits the ability to divide attention among several activities at once. All FSTs assess to some degree the extent of a person's impairment.

Estimates of impaired driving rates and alcohol-related traffic injuries and fatalities prompted the National Highway Traffic Safety Administration (NHTSA) in 1977 to commission the Southern California Research Institute (SCRI) to determine the best methods of detecting impaired drivers using field sobriety tests. An underlying premise was that better detection methods would lead to more impaired driving arrests, higher conviction rates and ultimately lower incidents of impaired driving.

The 1977 SCRI study validated earlier observations regarding the relationship between HGN and alcohol consumption and found that the HGN test, along with the WAT test, and the OLS test, were easy FSTs to at roadside and the most accurate in detecting impairment. Once the researchers identified the most accurate tests, they turned their attention to standardizing the administration of the tests in 1981. Through standardization, the SCRI researchers ensured that law enforcement officers everywhere could administer the tests quickly, easily, effectively, and uniformly. At that time, the researchers also found that when all three test results (HGN, WAT and OLS) were combined, it was possible to accurately determine whether an individual's BAC was 0.10 or higher eighty-three percent of the time.

After standardization, NHTSA funded a third study in 1983 to further corroborate these findings. Using data from the 1981 SCRI laboratory study, the NHTSA researchers determined that the HGN test was seventy-seven percent accurate in detecting whether an individual's BAC was 0.10 or higher. The WAT test was found to be accurate sixty-eight percent of the time. However, the NHTSA researchers found that when the results of the HGN and WAT test data were combined, the two tests were eighty percent accurate in detecting whether an individual's BAC was 0.10 or higher. Finally, the researchers predicted that the OLS test alone accurately indicated impairment sixty-five percent of the time. NHTSA researchers then conducted a field study and confirmed the tests' ability to “effectively discriminate between drivers with BACs less than 0.10% and drivers with BACs equal to or over 0.10%.” The field study also concluded that the HGN test was the most “powerful” of the three tests.

While these initial studies showed the accuracy of the HGN test, more recent studies demonstrate that the HGN test is even more accurate when administered by law enforcement officers trained and experienced in the administration of the HGN test. A 1986 study found the HGN test ninety-two percent accurate in detecting impairment. A 1987 study found that experienced law enforcement officers were correct ninety-six percent of the time in determining a 0.10 BAC or more using the HGN test.

The result of these studies was the standardized field sobriety test (SFST) battery used by law enforcement officers almost everywhere. The purpose of the SFST battery, and especially the HGN test, is to increase the ability of law enforcement to: (1) identify drivers with BACs in the 0.08-0.12 range that make up the bulk of the impaired drivers who do not necessarily exhibit exaggerated characteristics of impairment; and (2) detect impairment in alcohol-tolerant drivers who may not display any gross coordination and balance problems.

### Administering the HGN Test

The HGN test is very easy to administer. The law enforcement officer must administer the test in a way that ensures that the subject's eyes can be seen clearly, i.e., in a well lit area or by use of a flashlight to illuminate the subject's face. The subject should not face toward the blinking lights of a police cruiser or passing cars, which may cause optokinetic nystagmus. The subject does not have to be standing but can be sitting down. The officer informs the subject “I am now going to check your eyes.” The officer is not “testing” the subject's vision, as an ophthalmologist or optometrist would, but instead, the officer is “checking” the eyes for the physical manifestation of HGN.

Before checking the subject's eyes, the officer asks the subject to remove eyeglasses or inquires whether the subject is wearing hard or soft contact lenses. While the removal of the eyeglasses makes it easier for the officer to observe eye movement, glasses do not affect the HGN test results. Early concerns that contact lenses, especially hard contact lenses, may affect the HGN test result led some to provide for the subject to remove the lenses. However, contact lenses, hard or soft, do not affect the test in any way. While hard contact lenses may pop out when the eye moves as far to the side as it will go, officers are not taught to have subjects remove contact lenses. However, officers are taught to note whether the subject is wearing contacts and which type on the HGN Guide.

The officer also asks the subject whether he or she has any medical impairment that would either prohibit the subject from taking the test or that would affect the test results. The officer should note on the HGN Guide any condition that prohibits the taking of the test and then move on to the remaining SFSTs. If the subject claims to have a natural nystagmus or any other condition that may affect the test result, but does not prohibit the taking of the HGN test, the officer should note the condition but still perform the test.

The HGN test requires only an object for subjects to follow with their eyes, such as a pen or the tip of a penlight. The officer places the object approximately twelve to fifteen inches from the subject's face and slightly higher than eye level. Placing the object above eye level opens the subject's eyes further and makes their movement easier to observe.

The officer instructs the subject to follow the object with the eyes and the eyes only the head should remain still. If subjects have difficulty keeping their head still during the test, the officer is taught to have subjects hold their own head still by pressing the palms of their hands to their cheeks or to hold their own chin. The officer should try to avoid holding the subject's chin or using a flashlight as a chin rest because it brings the officer into contact with the subject and compromises officer safety. The officer then asks if the subject understands all the instructions.

After positioning the object, but before conducting the test, the officer checks for signs of medical impairment. First, the officer checks for “equal tracking” by moving the object quickly across the subject's entire field of vision to see whether the eyes follow the object simultaneously. The officer then checks for equal pupil size. Lack of equal tracking or equal pupil size may indicate blindness in one eye, a glass eye, a medical disorder or an injury. If the subject exhibits these characteristics, the officer should discontinue the HGN test and may need to seek medical assistance for the individual if a medical disorder or injury appears to exist.

While conducting the test, the officer looks for six “clues,” three in each eye that indicate impairment:
LACK OF SMOOTH PURSUIT—The officer moves the object slowly but steadily from the center of the subject's face towards the left ear. The left eye should smoothly follow the object, but if the eye exhibits nystagmus, the officer notes the clue. The officer then checks the right eye.
DISTINCT NYSTAGMUS AT MAXIMUM DEVIATION—Starting again from the center of the suspect's face, the officer moves the object toward the left ear, bringing the eye as far over as possible, and holds the object there for four seconds. The officer notes the clue if there is a distinct and sustained nystagmus at this point. The officer holds the object at maximum deviation for at least four seconds to ensure that quick movement of the object did not possibly cause the nystagmus. The officer then checks the right eye. This is also referred to as “end-point” nystagmus.
ANGLE OF ONSET OF NYSTAGMUS PRIOR TO FORTY-FIVE DEGREES—The officer moves the object at a speed that would take about four seconds for the object to reach the edge of the suspect's left shoulder. The officer notes this clue if the point or angle at which the eye begins to display nystagmus is before the object reaches forty-five degrees from the center of the suspect's face. The officer then moves the object towards the suspect's right shoulder. For safety reasons, law enforcement officers usually use no apparatus to estimate the forty-five degree angle. Generally, forty-five degrees from center is at the point where the object is in front of the tip of the subject's shoulder.

The officer also checks for vertical nystagmus. The officer checks for vertical nystagmus by raising the object several inches above the subject's eyes. Vertical nystagmus is not one of the HGN clues nor is it a part of the SFST battery. However, vertical nystagmus is a good indicator of high doses of alcohol, other central nervous system (CNS) depressants or inhalants, and the consumption of the drug phencyclidine (PCP). The officer should note the result and take precautions if vertical nystagmus is evident.

### Other Types of Nystagmus

There exist several non-alcohol related types of nystagmus caused by neural or muscle activity. These other types are due to a variety of causes, such as other vestibular system (inner ear) and nervous system disturbances and pathological disorders. Many times defendants will suggest that the nystagmus the law enforcement officer saw was actually caused by something other than alcohol or other drugs. However, a properly trained law enforcement officer will not mistake other types of nystagmus, natural or otherwise, with HGN when taking into account all of the facts that contribute to the arrest decision.

Nystagmus Caused by Non-Alcohol Related Disturbance of the Vestibular System: Rotational nystagmus is caused by a disturbance in the inner ear fluid when a person spins around. The nystagmus lasts only as long as the person is being spun. If an observer could see a person's eyes while that person was spinning, a distinct jerking of the eye would be evident. Post-rotational nystagmus occurs after the person stops spinning. The nystagmus lasts for several seconds and can easily be seen.

Caloric nystagmus is caused by the movement of the inner ear fluid due to a difference in temperature of the fluid between the left and right ear. One way this can occur is if warm water is poured in one ear and cold water is poured in the other. Obviously this is an implausible scenario at roadside.

Nystagmus Caused by Neural Activity: Some types of nystagmus are caused by neural or muscle activity. Optokinetic nystagmus occurs when the eyes fixate on an object that moves quickly out of sight or passes quickly through the field of vision, such as occurs when a subject watches utility poles pass by while in a moving car. Optokinetic nystagmus also occurs when the eyes watch an object displaying contrasting moving images, such as black and white spokes on a spinning wheel. In either case, because the nystagmus is caused by the eye trying to catch up with the moving object, it lasts only as long as it takes for the object to stop moving, for the object to pass out of the field of vision, or for the eye to catch up to the object. Epileptic nystagmus is also a jerk nystagmus caused by neural activity that occurs primarily during epileptic or other types of seizures.

In addition, some people will exhibit a slight eye tremor when the eye moves to maximum deviation. This tremor is due mostly to eye strain rather than to any type of alcohol impairment or medical condition. When the HGN test is administered properly, a law enforcement officer cannot confuse this eye tremor with HGN due to alcohol impairment for several reasons. First, the eye tremor lasts only briefly and law enforcement officers are taught to hold the eye at maximum deviation for at least four seconds to ensure that the jerking is sustained. Second, the officer is looking for a distinct nystagmus, not a slight eye tremor. And finally, distinct nystagmus at maximum deviation is only one clue among the three the officer is looking for when checking for HGN.

Nystagmus Due to Pathological Disorders: Nystagmus may occur in people with brain damage, brain tumors or inner ear diseases. These disorders and others like them occur in a small number of the general population and even less often in drivers. Many of these alternative causes are so severe that it is unlikely that persons afflicted with the disorders would be driving, would not know they have the disorder or would be unaware of the effect the disorder has on their body. In addition, these types of nystagmus may be pendular rather than jerk nystagmus.

Natural Nystagmus: The defense may argue that the nystagmus the law enforcement officer detected was actually a naturally occurring nystagmus rather than the result of alcohol impairment or any of the conditions listed above. As outlined below, the differences between any type of naturally occurring nystagmus and HGN are many and a properly trained officer will have no trouble distinguishing between the two at roadside.

Research indicates that a very small number of people exhibit a visible natural nystagmus. Those who have natural nystagmus generally know they have it and will most likely tell the officer before the test is administered. Visible natural nystagmus is evident only at particular angles of gaze, but not before or beyond that point. However, when administering the HGN test, the law enforcement officer is looking for not only nystagmus at a particular angle of gaze, but smooth pursuit and end-point nystagmus as well. Furthermore, in making the ultimate decision of whether the subject is impaired, the law enforcement officer is continually taking into account other facts, such as the subject's performance on the other SFSTs that suggest the subject is impaired by alcohol or other drugs. The law enforcement officer will never base an arrest decision solely on the results of the HGN test.

Physiological Nystagmus: Physiological nystagmus exists in every person's eye in order to keep the eye from tiring when fixated on one point. This nystagmus occurs so that light entering the eye will continually fall on non-fatigued cells of the retina. Physiological nystagmus cannot be seen with the naked eye and is controlled by a part of the brain system other than that affected by alcohol impairment. Because the officer can easily see HGN caused by alcohol with the naked eye, there is virtually no chance that a law enforcement officer could confuse physiological nystagmus with HGN.

The HGN test is designed to check the eyes for one type of nystagmus horizontal gaze nystagmus. Its results are not invalidated by virtue of the fact that other types or causes of nystagmus exist. As shown above, the various types of nystagmus manifest themselves in different ways. Law enforcement officers will not confuse HGN with any other type of nystagmus if the HGN test is conducted correctly. Research shows that the HGN test is a valid and reliable indicator of alcohol impairment and is the most effective roadside test for impaired drivers.

Accordingly, there is a need for for a portable observation device to perform certain tests in the field.

### SUMMARY

It is one objective of the observation device and method disclosed herein to provide a portable system for use in diagnosing some of the conditions listed above.

Assisted Diagnosis/Assisted Monitoring (ADAM), which is one embodiment of the observation method as disclosed herein, is accomplished as follows: The proposed system leverages state-of-the-art image processing algorithms to pinpoint and monitor upper body and facial features from multiple high-speed video streams. Based on the observed features, time series of many physiological measures are extracted, such as pupil size, eye movement (saccades, fixations, vergence, tremor), eyelid movement, head movement, facial expression, etc. Subsequently, these measures are compared against normative database values through signal analytic and statistical procedures. During this process, patient and task-specific parameters are taken into account to contextualize the reported diagnostic values and associated statistics. Different levels of ADAM are supported: on a low level, the value and estimated error of a single measure are available, while at a higher level, several different measures can be combined via a trained discrimination algorithm to estimate the probability of a specific disorder or condition.

Other objects and advantages of the present invention will, in part, be apparent from the specification when considered in conjunction with the drawings and claims hereof.

### BRIEF DESCRIPTION OF THE FIGURES

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limited of its scope, the invention will be described and explained with additional specificity and detail through the use of the accompanying drawings.

FIG. 1A is a user-side (or operator-side) view of the exemplary embodiment of the observation device in application mode.

FIG. 1B is a subject-side view of the exemplary embodiment of the observation device in application mode.

FIG. 1C is a side view of the exemplary embodiment of the observation device in transport mode.

FIG. 1D is two perspective views (user/operator side and subject side) of the exemplary embodiment of the observation device in application mode.

FIG. 1E is a subject-side of another exemplary embodiment of the observation device in application mode.

FIG. 1F is a side view of another exemplary embodiment of the observation device in transport mode.

FIG. 2A is a perspective view of a first embodiment of the observation device and a subject during application.

FIG. 2B is a perspective view of a second embodiment of the observation device and a subject during application.

FIG. 2C is a perspective view of a third embodiment of the observation device and a subject during application.

FIG. 3 is a general schematic representation of the system components for the exemplary embodiment of the observation device.

FIG. 4 is a general schematic representation of the interaction between the subject and the exemplary embodiment of the observation device.

FIG. 5 is a graphical representation of the observation device as used to monitor the evolution of a subject's condition.

DETAILED DESCRIPTION - LISTING OF ELEMENTS

| ELEMENT DESCRIPTION | ELEMENT # |
|---|---|
| Observation device | 10 |
| Subject | 12 |
| Subject's eye | 12a |
| Subject's head | 12b |
| Subject's ear | 12c |
| Base member | 20 |
| Illuminator | 22 |
| Image collector | 24 |
| Transport engagement notch | 25 |
| Application engagement notch | 26 |
| Handle member | 30 |
| Stimulus generator | 31 |
| User interface | 32 |
| Feedback generator | 34 |
| Transport engagement tab | 35 |
| Application engagement tab | 36 |
| Projection system | 40 |
| External CPU | 42 |
| Data transmission line | 44 |
| External visual stimulus | 46 |

### DETAILED DESCRIPTION

Before the various embodiments of the present invention are explained in detail, it is to be understood that the invention is not limited in its application to the details of construction and the arrangements of components set forth in the following description or illustrated in the drawings. The invention is capable of other embodiments and of being practiced or of being carried out in various ways. Also, it is to be understood that phraseology and terminology used herein with reference to device or element orientation (such as, for example, terms like “front”, “back”, “up”, “down”, “top”, “bottom”, and the like) are only used to simplify description of the present invention, and do not alone indicate or imply that the device or element referred to must have a particular orientation. In addition, terms such as “first”, “second”, and “third” are used herein and in the appended claims for purposes of description and are not intended to indicate or imply relative importance or significance.

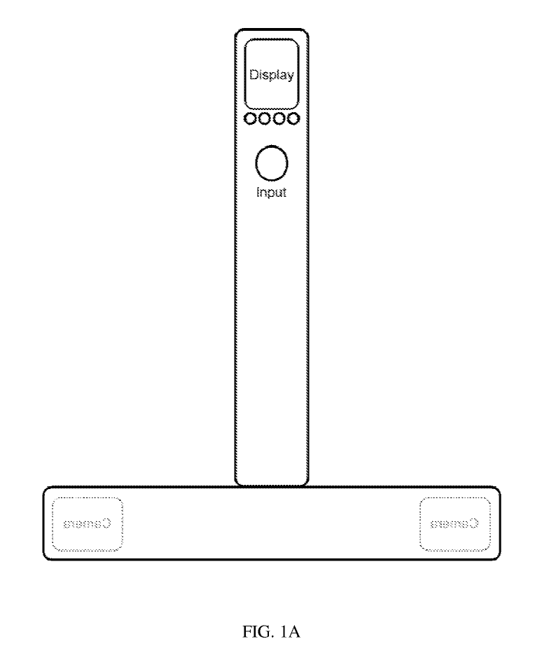

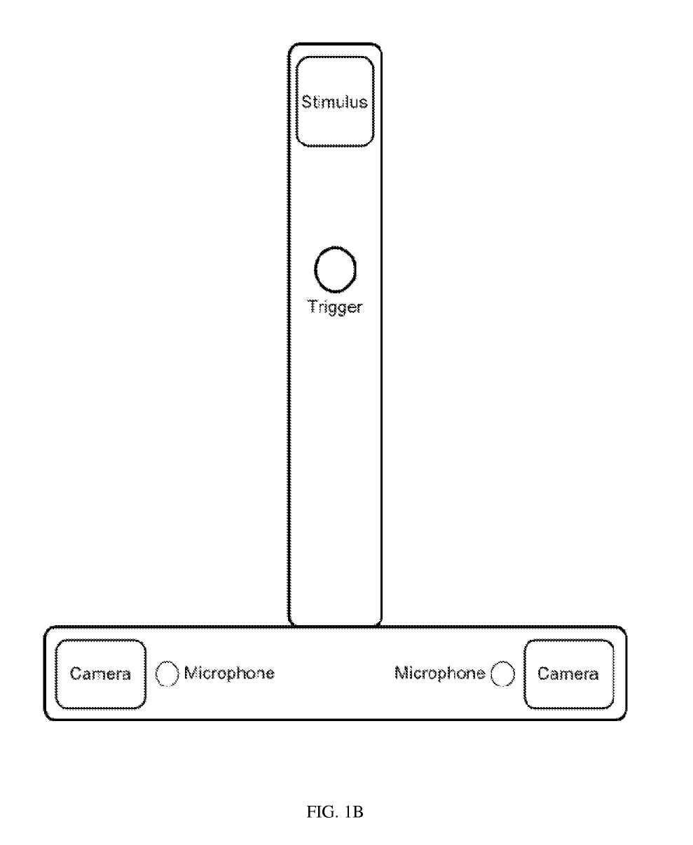

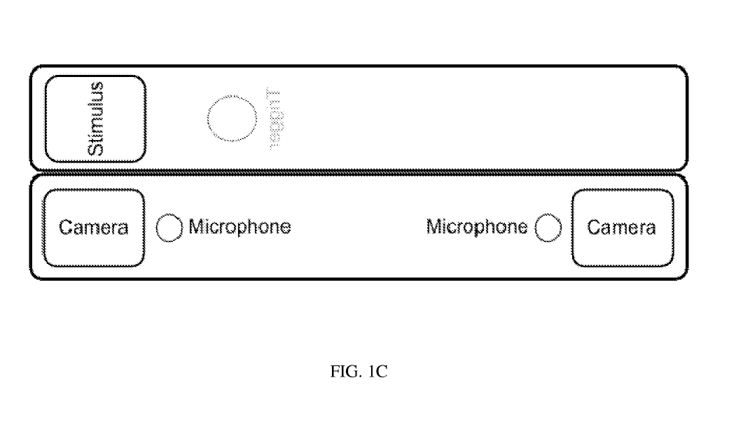

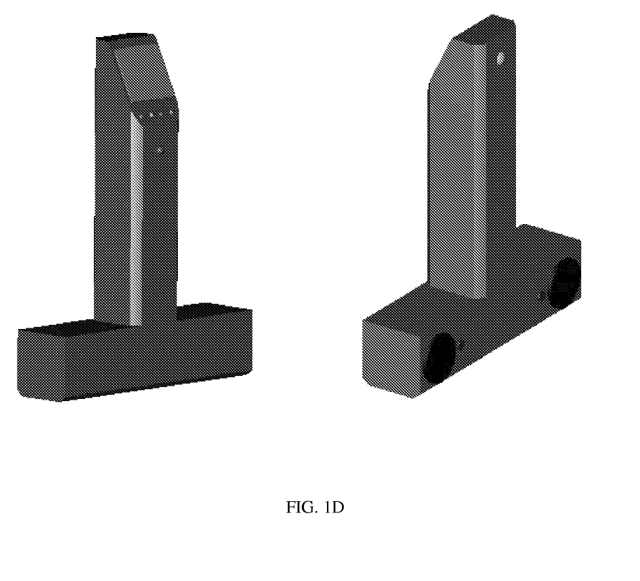

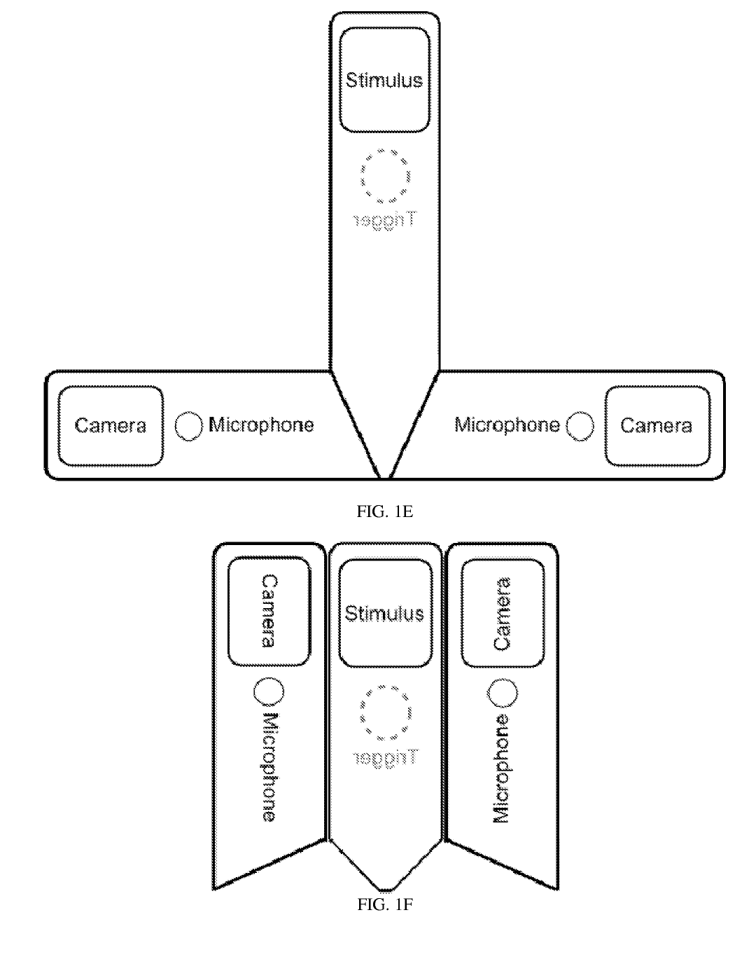

Referring now to FIGS. 1A-1C, an exemplary embodiment of an observation device 10 is disclosed. The exemplary embodiment of the observation device 10 is handheld and ultra-portable so that it may be used in the field in emergency situations, such as by emergency medical technicians, armed services medics, firefighters, etc. Accordingly, it is contemplated that the exemplary embodiment as shown in FIGS. 1A-1C will be especially useful in an emergency setting. Additionally, the exemplary embodiment of the observation device 10 may be configured to measure, observe, and/or record the three major modalities using ultrasonography, electromagnetic spectrum information (e.g., video images), and electrophysiology.

Generally, the exemplary embodiment of the observation device 10 includes two sides—a subject side that is generally positioned facing the subject during observation and a user side that is generally positioned facing the user during operation. As shown in FIG. 1A, the user or operator side of the observation device 10 in the exemplary embodiment includes a user interface 32 that may include a feedback generator 34. Through the user interface 32, the user is able to operate the observation device 10 and control various functions thereof, which are described in detail below. The feedback generator 34 may be used to alert the user to certain conditions and/or analyses based upon observations of the observation device 10. Those conditions may be psychological, physiological, physical, psycho-physiological and/or combinations thereof

The subject side of the observation device 10 in the exemplary embodiment (best shown in FIG. 1B) includes at least one image collector 24, at least one stimulus generator 31, a trigger, and at least one audio collector 28. Although shown on the handle member 30, the stimulus generator 31 may be positioned on the base member 20, or the base member 20 may include one or more stimulus generators 31 and the handle member 30 may include one or more stimulus generators 31. The stimulus generator 31 may be a visual, audio, olfactory, or any other type of stimulus generator that elicits a response in the subject. The specific type of stimulus generator 31 used with the observation device 10 will depend on the specific application for the observation device 10 and the types of tests it is designed to employ. Accordingly, the stimulus generator 31 or number thereof in no way limits the scope of the observation device 10. Furthermore, depending on the type of test and/or application for the observation device 10, or the condition that the user desires to detect and/or diagnose, the user interface 32 may allow the user to change the frequency, duration, or pattern of visual and/or aural stimuli. Different stimuli may be provided simultaneously for optimal diagnosis of different subject conditions. The subject side of the observation device may also include one or more illuminators 22 to provide for increased clarity and/or precision with the image collector 24.

As is apparent from a comparison of FIGS. 1A and 1B to 1C, the exemplary embodiment of the observation device 10 includes an application and a transport configuration, which results in the extreme portability of the exemplary embodiment of the observation device 10. The exemplary embodiment includes a base member 20 and a handle member 30, which are oriented parallel to one another when the observation device 10 is configured in the transport mode (best shown in FIG. 1C). In the transport mode, the observation device 10 may easily be moved from one location to another, and the base member 20 and handle member 30 are secured to one another by any structure or method known to those skilled in the art.

In the application mode, as shown in FIGS. 1A and 1B, the base member 20 and the handle member 30 are oriented perpendicular to one another. In this mode, the base member 20 and handle member 30 are in communication with one another, either wired or wirelessly. As with the transport mode, in the application mode the base member 20 and handle member 30 may be secured to one another by any structure or method known to those skilled in the art. For example, corresponding notches and tabs, slots and rails, Velcro, etc. may be used to secure the base member 20 to the handle member 30.

For most tests/diagnosis methods for which the exemplary embodiment of the observation device 10 will be used, it is contemplated that the observation device itself will be in motion for at least a portion of the test. Although not shown, a position sensing device may be included in any embodiment of the observation device 10. A position sensing device may be used to account for movements of the observation device 10 during testing and/or analysis of the subject. The position sensing device may be an accelerometer, gyroscope, or any other device known to those skilled in the art that is capable of providing a relative and/or absolute position or path of travel of an object. The inclusion of a position sensing device may be especially useful in embodiments of the observation device 10 configured for performing a smooth pursuit test of the subject's eyes 12a. The observation device 10 may also be configured to include a geo-tracking or geo-tagging member using elements, structures, and/or methods known to those skilled in the art, such as global positioning satellites.

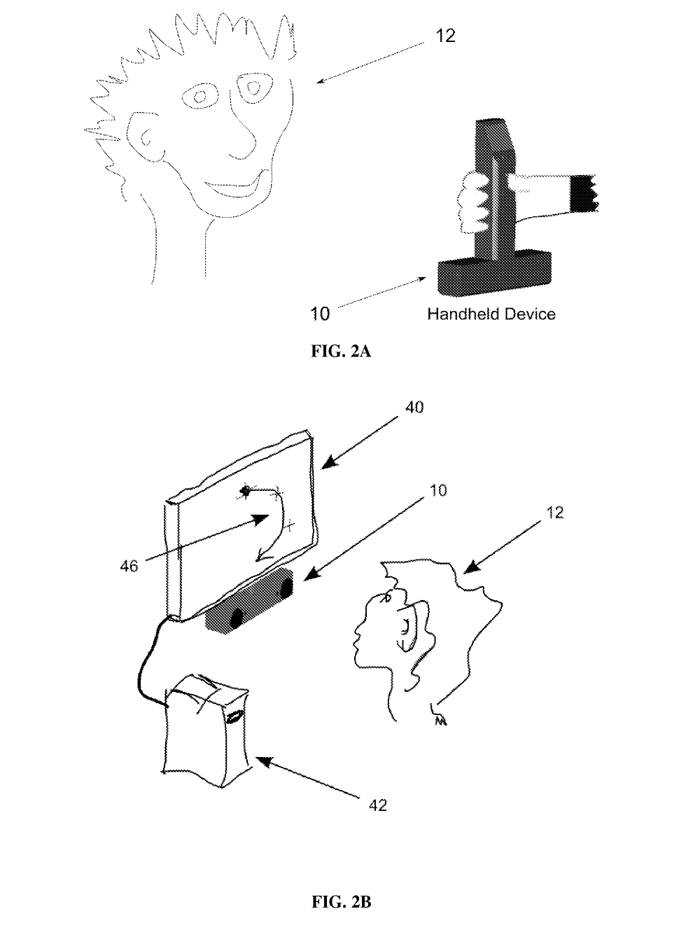

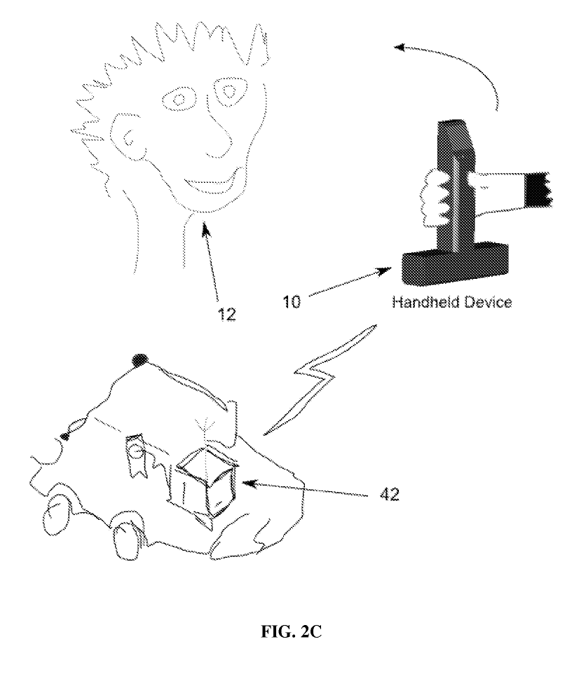

The exemplary embodiment of the observation device 10 is shown in the application mode with a subject in FIG. 2A. As shown, the exemplary embodiment is handheld and may easily be moved from one position to another. As the position of the observation device 10 moves, the position sensing device will determine and/or record the precise position of the observation device 10 in three-dimensional space so that the movements of the subject 12 attributable to the movements of the observation device 10 may be accounted for during analysis of the data.

A second embodiment of the observation device 10 is shown in FIG. 2B. In this embodiment, the base member 20 is in a fixed position and is shown mounted to a projection system 40. It is contemplated that this embodiment will be most useful for clinical and/or home settings. In this embodiment an external central processing unit (CPU) is in operative communication with the base member 20 via a data transmission line 44, which may not be required in wireless embodiments. The base member 20 and the external CPU 42 cooperate to produce a visual stimulus 46 on the projection system 40. As the visual stimulus 46 is displayed, the image collectors 24 in the base unit 20 observe and/or record various parameters of the subject 12. The parameters that the image collector records are dependent on the tests with which the observation device 10 is designed to be used and/or the conditions for which the subject is being tested. As with the exemplary embodiment, the second embodiment may include additional types of stimulus generators, such as auditory and/or olfactory, different types of response collectors (such as microphones), and different user interfaces 32 and/or feedback generators 34. Depending on the embodiment of the observation device 10, it may be configured to administer a specific test (e.g., by providing a stimulus to the subject and recording the response and/or feedback to that stimulus) or it may be configured to observe certain parameters based on a test primarily administered by the user.

If the base member 20 includes an appropriately configured integrated processing unit, an external CPU 42 will not be required to perform tests using the observation device 10. However, in certain embodiments an external CPU 42 will be required for the observation device to administer certain tests. For example, if the base member 20 does not include a graphics adaptor, an external CPU 42 will be required to provide visual stimulus 46, and if the base member 20 does not include an auditory function in the stimulus generator 31, then some external components will be required to provide auditory stimulus, etc.

The second embodiment of the observation device 10 may be used with components other than a base member 20 as long as the observation device 10 includes at least one component capable of observing a relevant parameter for the test the observation device 10 is designed to administer. For example, if an image collector 24 is in operative communication with a storage media or processing unit, that image collector 24 may be used in conjunction with an external CPU 42 and projection system 40 to provide the user with the appropriate data for a specific application.

A third embodiment of the observation device 10 is shown in FIG. 2C. In this embodiment, a CPU is external to the observation device 10 and communicates therewith either wired or wirelessly. The CPU may be used to record the data that the observation device 10 is observing, and may also be used to analyze the data to provide for real-time feedback and diagnosis. The observation device 10 in the third embodiment may also include a processing unit and a storage media, but it is contemplated that the analysis function will be performed by the external CPU 42.

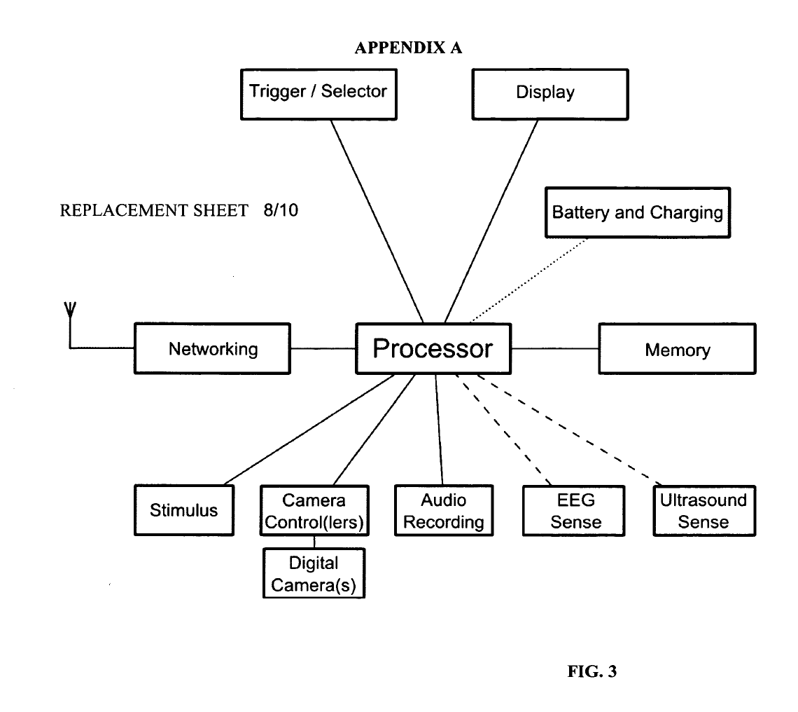

A general schematic of the various elements of the observation device 10 that may be used in a specific embodiment thereof is shown in FIG. 3. As is readily apparent from FIG. 3, nearly all functions of the observation device 10 are in some type of communication with a processing unit, either internal to the observation device or external thereto. However, in other embodiments, certain functions/elements may operate independently of the processing unit. For example, in one embodiment a flashlight or other visual display may provide the stimulus and the observation device will simply include an image collector and electronic storage media. In another embodiment, a speaker may be used to generate an auditory stimulus, or it may be user-generated, and the observation device will simply include an image collector and/or feedback collector (e.g., microphone), and/or electronic storage media.

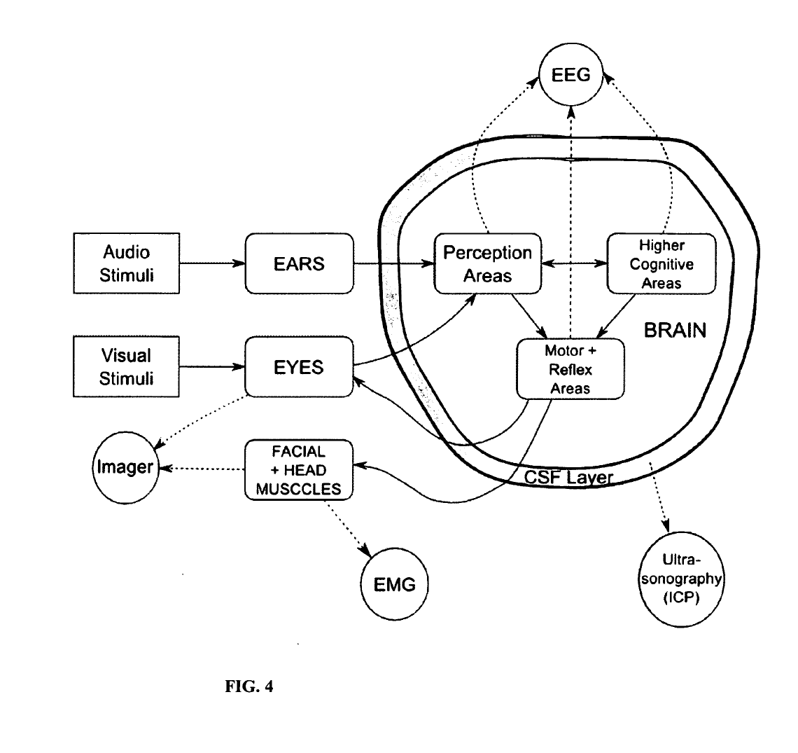

A general schematic of the interaction between the subject 12 and the observation device 10 is shown in FIG. 4. The integration of electrophysiology and ultrasonography into the observation device 10 is also shown in FIG. 4. As shown, the observation device 10 may be used to provide audio and/or visual stimuli, which stimuli are then processed by the subject 12. Electrophysiology in the form of electroencephalography may be used to provide values of other parameters during the subject's subjection to and/or processing of the stimuli, as may ultrasonography. The stimuli may generate a specific response or series of responses from the subject 12, which may be manifested through facial, head, and eye muscles. The observation device 10 may be used to observe and/or record those manifestations. Electrophysiology in the form of electromyography may be used to observe and/or record additional features/characteristics of the subject 12 during manifestations resulting from the stimuli.

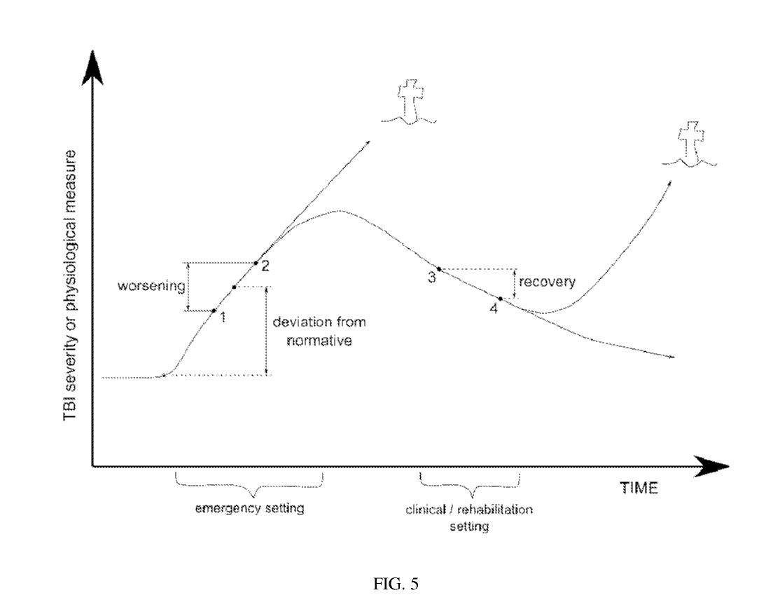

A graphical representation of TBI over time is shown in FIG. 5. Although the diagnosis and recovery rate for a subject suffering from TBI is but one of many conditions for which the observation device 10 may be used, TBI provides a general example for how the observation device 10 and data accumulated therefrom may be used in different settings.

As shown in FIG. 5, the x-axis represents time and the y-axis represents severity of TBI (or more generally, the condition and/or physiological parameter observed). The beginning of the curved line is marked with an X, which represents the point in time in which the subject's system has been insulted in some manner (e.g., head impact, subjection to electromagnetic radiation, etc.). From this point in time forward, the observation device 10 may be used to observe and/or record a specific physiological parameter and correlate that parameter to severity of a certain condition.

From the point labeled “1” to the point labeled “2” in FIG. 5, the severity of the subject's 12 TBI is increasing, as the slope of the line is positive. By comparing the value of the parameter observed on the subject 12 to a normative value for that parameter, the user may determine the severity of the tested condition, wherein the normative value is represented by the dashed horizontal line tangent to the X. As shown in FIG. 5, the severity of the condition may increase, and if left untreated or unmitigated eventually lead to death. However, if properly diagnosed and treated, the severity of the condition may decrease over time, as shown by the portion of the curve in FIG. 5 with a negative slope.

It is contemplated that the part of the line with a positive slope will generally encompass the emergency setting and the majority of the portion of the line with a negative slope will generally encompass the clinical or rehabilitation setting. During the clinical or rehabilitation setting, the observation device 10 may be used to determine the amount and/or rate of recovery of the subject 12 by comparing the observed parameter to previously observed values thereof, or to a normative value therefore. Furthermore, the parameter observed may be used to determine the severity of a condition rather than the parameter itself being directly correlated to a specific condition and/or severity thereof. As shown in FIG. 5, from the point labeled “3” to the point labeled “4” on the curved line, the subject's 12 condition is improving over time.

Typically, the observation device 10 will be configured to observe the subject's eye 12a and/or the position or rate of change of position thereof, the subject's head 12b and/or the position or rate of change of position thereof, the subjects facial features (e.g., brow, cheek, chin, lip, and/or jaw position) and/or the rate of change thereof, and the subject's pupil size and/or rate of change thereof. The observation device 10 may also be configured to measure the difference in pupil size between one eye and the other eye on the same subject, or the difference in rate of change between the two. The observation device 10 may also be configured to measure the difference in tracking between the two eyes of one patient, the eyelid movement for each eye and the rate of that movement, and the eyebrow shape, movement, and rate of movement. Furthermore, the observation device 10 may be configured to measure the shape of the subject's 12 mouth, lips, and/or movements thereof as well as micro and macro expressions of the subject 12. Head and torso positions and expressions of the subject 12 may also be measured with the observation device 10, such as head tilting, shoulder shrugs, head nodding, head or shoulder tremors, etc.

As mentioned above, the observation device 10 may be configured with microphones. Microphones or other feedback collectors may be used to observe the subject's feedback to certain stimuli or commands. For example, microphones may be used to track the subject's 12 breathing pattern or response to specific commands (i.e., “repeat after me”). The observation device 10 also may be configured with an infrared camera for tracking blood flow changes in the subject's 12 face, head, and/or neck.

Any embodiment of the observation device 10 may be configured to provide nearly an infinite number of stimuli. For example, lights, series of lights, patterns of lights, colors of lights, or any other visual stimulus including a combination thereof and/or animation thereof may be used depending on the specific embodiment of the observation device 10. The visual stimulus may vary in frequency, amplitude, intensity, color, etc. depending on the test the observation device 10 is designed to administer. Furthermore, the observation device 10 may be configured to provide any type of auditory, olfactory, and/or tactile stimulus (e.g., a puff of air). Any of these various stimuli may be employed simultaneously or in a predetermined sequence depending on the specific test with which the observation device 10 is used. Any method or apparatus that serves to stimulate the central nervous system in any manner may be used with the observation device 10, whether integrated therewith or separately embodied.

Any embodiment of the observation device 10 may be configured to provide nearly an infinite number of types of feedback to the user. For example, a series of lights or lights of different colors and/or audible signal may be used to alert the user when he or she is using the observation device 10 in an improper manner (e.g., moving the observation device 10 too rapidly as determined by an integrated position sensing device in communication with a processing unit). Alternatively, the observation device 10 may be configured to provide tactile feedback to the user during use, such as vibrations in the handle thereof

Any embodiment of the observation device 10 may also include a power source. The power source may be battery, kinetic solar, or a combination thereof. The optimal type and size of a power source will vary depending on the specific embodiment of the observation device 10 and the power requirements thereof.

Furthermore, any embodiment of the observation device 10 may be equipped with on-board processor, or with a storage media for storing the information for later processing and/or analyzing. Any embodiment may also include a communication device, which may be wired or wireless. Any embodiment of the observation device 10 also may be configured to provide immediate feedback to the user and/or subject 12 through a video and/or graphical display. The observation device 10 may also be used to record the data upon which the user has based a diagnosis to provide validation for the diagnosis.

In another exemplary embodiment show in FIGS. 1E and 1F, the observation device 10 includes to fold-down arms that are pivotally attached to a main body. Each fold down arm includes an image collector, and may also include a feedback collector, an illuminator 22, an audio collector 28 (such as a microphone), and a stimulus generator 31. It is contemplated this embodiment would be quite similar in operation to the exemplary embodiment pictured in FIGS. 1A-1D. However, in this embodiment to convert the observation device 10 from the transport mode (shown in FIG. 1F) to the application mode (shown in FIG. 1E), the user simply folds the arms away from the main body so that the observation device 10 is oriented in a T-shape, wherein the arms are substantially perpendicular to the main body. Accordingly, to convert the observation device from the application mode to the transport mode, the user simply folds the arms in so that they are adjacent the main body and substantially parallel thereto. An infinite number of other orientations of the observation device 10 exist and will become apparent to those skilled in the art in light of the present disclosure. Accordingly, the precise angles, distances, orientations, configurations, etc. of the illuminator 22, image collector 24, audio collector 28, feedback collector, stimulus generator 31, user interface 32, feedback generator 34, and/or number thereof in no way limits the scope of the observation device 10 as disclosed and claimed herein.

The materials used to construct the observation device 10 and various electrical components thereof may be any suitable material known to those skilled in the art that is suitable for the particular application of the observation device 10. For example, various structural components of the observation device 10 may be constructed of polymers, cellulosic materials, metal, metal alloys, and/or combinations thereof.

It should be noted that the present disclosure is not limited to the specific embodiments pictured and described herein, but is intended to apply to all similar apparatuses and methods for observing physiological and/or anatomical conditions. Modifications and alterations from the described embodiments will occur to those skilled in the art without departure from the spirit and scope of the present disclosure.

1. An observation device comprising:
a. a base member, wherein said base member is portable and includes an image collector; and
b. a processing unit, wherein said processing unit is operable to derive at least one metric from an image collected by said image collector.

2. An observation device comprising:
a. a base member, said base member comprising:
i. at least one image collector;
ii. at least one illuminator;
iii. a processor unit;
b. a handle member, said handle member comprising at least one stimulus generator.

3. The observation device according to claim 2 wherein said handle member further comprises a user interface.

4. The observation device according to claim 3 wherein said handle member further comprises a user input.

5. The observation device according to claim 4 wherein said user interface is further defined as including a user input and a user feedback generator.

6. The observation device according to claim 2 further comprising a storage media positioned in either said handle member or said base member.

7. The observation device according to claim 2 further comprising an electronic communication interface positioned in either said handle member or said base member.

8. The observation device according to claim 2 further comprising a position sensing component for sensing the position of said stimulus generator.

9. The observation device according to claim 2 further comprising a position sensing component for sensing the position of said image collector.

10. The observation device according to claim 1 further comprising a component for observing electrophysiology.

11. The observation device according to claim 1 further comprising a component for performing ultrasonography.

12. A method for observing a subject comprising:
a. providing a stimulus to said subject;
b. observing the subject's response to said stimulus; and
c. correlating the subject's response to a condition of said subject.

13. The method according to claim 12 wherein said stimulus is further defined as a visual stimulus.

14. The method according to claim 13 wherein a handheld observation device is used to provide said stimulus.
